(19) United States
(12) Patent Application Publication
(10) Pub. No.: US 2007/0264239 A1
(43) Pub. Date: Nov. 15, 2007

(54) ISOLATION OF PERICYTES

(75) Inventors: Johnny Huard, Wexford, PA (US); Bruno M. Peault, Munhall, PA (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignee: University of Pittsburgh - Of the Commonwealth System of Higher Education, Pittsburgh, PA (US)

(21) Appl. No.: 11/746,979

(22) Filed: May 10, 2007

Related U.S. Application Data
(60) Provisional application No. 60/799,195, filed on May 10, 2006.
(60) Provisional application No. 60/799,430, filed on May 10, 2006.

(51) Int. Cl. C12N 5/08

(52) U.S. Cl. 424/93.7; 435/366; 435/325

(57) ABSTRACT

In one embodiment, the invention provides a pericyte having a marker pattern comprising CD146+, CD34−, and CD45−, wherein said pericyte is substantially isolated from cells that are CD146− or CD31+ or CD34+ or CD45+ or CD56+ or NG2− or CD133−. The invention also provides populations of such pericytes. In another embodiment, the invention provides a method for isolating a pericyte. In another embodiment, the invention provides a method for modeling tissue in vivo.

### CROSS-REFERENCE TO RELATED APPLICATIONS

This patent application claims the benefit of U.S. Provisional Patent Application Nos. 60/799,430 and 60/799,195, both of which were filed May 10, 2006, and both of which are incorporated by reference.

### STATEMENT REGARDING FEDERALLY SPONSORED RESEARCH AND DEVELOPMENT

This invention was made in part with Government support under Grant Numbers R01-AR049684; RO1-DE13420-06; IU54AR050733-01 awarded by the National Institutes of Health and under Department of Defense Grant W81XWH-06-1-0406. The Government may have certain rights in this invention.

### BACKGROUND OF THE INVENTION

Multipotent and pluripotent stem cells, particularly mesenchymal stem cells (MSCs) have been proposed as reagents that can facilitate methods for engineering the generation, growth and healing of disparate tissue types (tissue modeling). However, obtaining a substantially homogenous population of multipotent or pluripotent stem cells is difficult. Stem cells are rare, and it can be difficult to identify stem cells among the other types of cells from which they can be isolated (e.g., bone marrow, adipose tissue, etc.). Accordingly, improved methods for identifying and isolating pluripotent cells are desired.

### BRIEF SUMMARY OF THE INVENTION

In one embodiment, the invention provides a pericyte having a marker pattern comprising CD146+, CD34−, and CD45−, wherein the pericyte is substantially isolated from cells that are CD146− or CD31+ or CD34+ or CD45+ or CD56+ or NG2− or CD133−. The invention also provides populations of such pericytes.

In another embodiment, the invention provides a method for isolating a pericyte, the method comprising obtaining tissue from a donor, dissociating cells within said tissue, assaying for a pericyte which is CD146+, CD34−, and CD45−, and culturing the pericyte.

In another embodiment, the invention provides a method for modeling tissue comprising obtaining tissue from a donor source, dissociating cells within the tissue, assaying for a pericyte which is CD146+, CD34−, and CD45−, and introducing the pericyte into a recipient subject at a location for the pericyte to generate or repair tissue within the recipient subject.

These aspects and additional inventive features will become apparent from the following detailed description.

### DETAILED DESCRIPTION OF THE INVENTION

In one embodiment, the invention provides a method for isolating a mammalian pericyte. In accordance with the method, tissue is first obtained from a donor source, which is typically a mammal and most preferably a human donor. The donor can be but need not be alive at the time of the donation and can be a cadaver donor, so long as the tissue from which the pericyte is obtained contains live pericytes. Moreover, the donor can be an embryo or placental tissue.

The tissue derived from the donor source then is processed so that the cells within the tissue become dissociated sufficiently for facilitating cell sorting. For example, the tissue can be cut into small pieces and then treated enzymatically (e.g., with trypsin, chymotripsin, dispase, etc.) to facilitate dissociating single cells from the gross tissue sample.

Following dissociation, the cells are assayed for a pericyte-specific expression pattern: positive for CD146 (i.e., CD146+), not expressing CD34 (i.e., CD34−), and not expressing CD45 (i.e., CD45−). The cells also can be assayed for NG2, as pericytes are positive for NG2 (i.e., NG2+). The cells also can be assayed for CD133, as pericytes are positive for CD133 (i.e., CD133+). Also, pericytes can be further identified as not expressing CD56 (i.e., CD56−), and/or not expressing CD31 (i.e., CD31−) and/or not expressing CD144 (i.e., CD144−) and/or not expressing vWF (i.e., vWF−) and/or not expressing the Ulex europaeus ligand.

Cells exhibiting the pericyte-specific expression pattern thereafter can be isolated or separated from cells not displaying the pericyte-specific expression pattern. Preferably, the pericyte is substantially free of other cell types (e.g., adipocytes, myocytes, red blood cells, other stromal cells, etc.) and extracellular matrix material; more preferably, the pericyte is completely free of such other cell types and matrix material.

Any suitable method can be employed to assay for the pericyte-specific expression pattern and to isolate or separate the pericytes from the other cells and tissue material. Immunohistochemical techniques are conveniently employed to identify the pericytes, as antibodies for all of the markers are commercially available and otherwise can be generated by standard techniques. Flow cytometry can be employed as well for sorting the pericytes and separating them from the other cells. A preferred technique employs fluorescence activated cell sorting (FACS). If desired, the sorted cells can be re-analyzed and RT-PCR performed to verify the absence of contaminant cells.

Once the pericytes have been separated from the other cells, they can be cultured. Any suitable culture conditions can be employed. Moreover, as indicated in the Examples below, pericytes can be cultured and expanded for many months (e.g., more than one month, or more than two, three, four, or five months). Accordingly, another aspect of the present invention provides a mammalian pericyte having a marker pattern comprising CD146+, CD34−, and CD45−, wherein said pericyte is substantially isolated from cells that are CD146− or CD31+ or CD34+ or CD45+ or CD56+ or NG2− or CD133− or CD144+ or a combination of any or all of these markers. The pericyte can alternatively be CD31−, CD56−, NG2+, CD133+, CD144− or any combination of these additional markers.

Desirably, the pericyte is within a substantially homogenous population of like cells. The population can comprise, for example, at least about 10 pericytes, such as at least about 25 pericytes, and more typically comprises at least about 100 or at least about 500 pericytes. It is possible for the inventive population to comprise at least about 1000, such as at least about 2000, at least about 3000, at least about 4000 or even at least about 5000 pericytes. The population can comprise greater than these numbers, depending on culture conditions. Indeed, for medical or veterinary applications, a population of pericytes can be concentrated from several cultures, if it is desired to introduce a large number of such cells into an individual.

One property of pericytes that facilitates their medical or veterinary application is their pluripotency. In this respect, the inventive pericyte can have one or more mesodermal developmental phenotypes. For example, such developmental phenotypes can include adipogenic, chondrogenic, myogenic, myocardiogenic, neurogenic, odontogenic, osteogenic, and vascular developmental phenotypes. More preferably, the pericyte has two or more of such developmental phenotypes. Thus, the pericytes can develop into adipocytes or preadipocytes (e.g., committed adipose precursors), chondrocytes or prechondrocytes (e.g., committed cartilage precursors), myocytes or myotubules or committed muscle precursor cells, myocardial cells, neurons and neural-like cells, odontocytes or committed odontoprecursor cells, osteocytes or committed osteocyte precursor cells, or vascular/endothelial cells.

Assaying the developmental potential of the pericytes can be achieved by exposing them to suitable medium for inducing differentiation in vitro and then assaying for phenotypic hallmarks of the differentiated tissue or committed precursors. For example, the pericytes can be cultured in the presence of, or in media conditioned by, cells of the respective type to be differentiated. Alternatively, the pericytes can be cultured in the presence of agents (e.g., growth factors, cytokines, extracellular matrix material, etc.) known to prompt differentiation of pluripotent cells along a desired developmental pathway. Alternatively, the pericytes can be xenotransplanted into an animal host under conditions for them to differentiate in vivo. Thereafter, the animal can be sacrificed or a tissue sample excised from the animal, which can be assayed for cells of the pericyte species. For example, a mouse host can be injected with human pericytes and the mouse's tissue can later be examined for the presence of human cells, which can be assayed to determine whether they have differentiated. For example, antibodies recognizing PPARγ and leptin can be employed to assess adipogenic (PPARγ+, leptin+) differentiation. Antibodies recognizing NF, TOH, and CNPase can be used to assess neural (NF+, TOH+, CNPase+) development. Antibodies specific for cardiac troponin I, atrial natriuretic peptide (ANP), Nkx2.5, α-myosin heavy chain (α-MHC), GATA-4, and connexin43 can be used to investigate the acquisition of a myocardiac phenotype by the injected cells. Capillary density and antibodies recognizing vWF, CD144, CD34 and CD31 and the expression of VEGF and VEGF receptor (KDR) can be used to assess vascular development. Markers for these and other differentiation pathways are known to those of skill in the art.

Because the inventive pericytes have a developmental phenotype, they can be employed in tissue engineering. In this regard, the invention provides a method of producing animal matter comprising maintaining the inventive pericytes under conditions sufficient for them to expand and differentiate to form the desired matter. The animal matter can include mature tissues, or even whole organs, including tissue types into which the inventive pericytes can differentiate. Typically, such animal matter will comprise fat, muscle (e.g., smooth muscle or myocardium), cartilage, bone, vasculature tissues, and the like. More typically, the animal matter will comprise combinations of these tissue types (i.e., more than one tissue type). For example, the animal matter can comprise all or a portion of an animal organ or a limb (e.g., a leg, a wing, an arm, a hand, a foot, etc.). Of course, in as much as the pericytes can divide and differentiate to produce such structures, they can also form anlagen of such structures. At early stages, such anlagen can be cryopreserved for future generation of the desired mature structure or organ.

To produce such structures, one or more pericytes can be isolated as discussed herein and directly introduced into a recipient subject (e.g., a human or veterinary patient or a laboratory test animal) at a location for the pericyte to generate or repair tissue within the recipient subject. However, the donor and source need not be the same individual or the same species. In fact, the source can be an embryo or placental tissue. Alternatively, pericytes that have been cultured (e.g., after one or several passages) can be introduced into the recipient subject at a location for the pericyte to generate or repair tissue within the recipient subject. The potential for an immune reaction against the pericytes can be reduced if the source of the pericytes is the same individual as the recipient subject (i.e., autotransplantation). In this sense, the inventive method can be used to concentrate an individuals' pericytes, which can then be reintroduced at a desired site for tissue modeling or generation. As the pericytes retain their capacity for differentiation even after prolonged culturing, if the number of pericytes harvested from an individual is too low, they can be expanded in culture and then implanted.

Thus, for example, the invention can facilitate the regeneration of tissues (e.g., bone, muscle, cartilage, tendons, adipose, etc.) within the recipient subject where the pericytes or populations of pericytes are implanted into such tissues. In other embodiments, and particularly to create anlagen, the pericytes can be induced to differentiate and expand into tissues in vitro. In such applications, the pericytes can be cultured on substrates that facilitate formation into three-dimensional structures conducive for tissue development. Thus, for example, the pericytes can be cultured or seeded onto a bio-compatible lattice, such as one that includes extracellular matrix material, synthetic polymers, cytokines, growth factors, etc. Such a lattice can be molded into desired shapes for facilitating the development of tissue types. Also, at least at an early stage during such culturing, the medium and/or substrate can be supplemented with factors (e.g., growth factors, cytokines, extracellular matrix material, etc.) that facilitate the development of appropriate tissue types and structures. Indeed, in some embodiments, it is suitable to co-culture the pericytes with mature cells of the respective tissue type, or precursors thereof, or to expose the pericytes to the respective conditioned medium, as discussed herein. Thereafter, the differentiated or partially-differentiated pericytes (including any lattice material) can be implanted into the recipient subject.

The following examples further illustrate the invention but, of course, should not be construed as in any way limiting its scope.

### EXAMPLE 1

This example demonstrates the potential of sorted human pericytes to generate muscle tissue. These results demonstrate that sorted pericytes cultured in muscle proliferation medium, and then in muscle fusion medium, developed into multinucleated myotubes expressing myosin heavy chain. Furthermore, sorted pericytes (CD146+/CD45−/CD34−/CD144−/CD56−), myoblasts (CD146−/CD45−/CD34−/CD144−/CD56+), and unseparated muscle cells all regenerated muscle fibers after injection into the cardiotoxin-injured skeletal muscles of SCID-non-obese diabetic mice, indicating a muscle-regenerating potential for pericytes. Furthermore, the myogenic capacity of these pericyte cultures was retained after up to five months of culture in vitro. The following methodology was employed:

Human tissues. First-trimester human embryos were obtained anonymously, with the approval and according to the guidelines of the French Comité National d'Ethique and Comite pour l'Ethique dans les Sciences de la Vie, following voluntary pregnancy interruptions performed with the RU486 anti-progestative compound. Developmental stages were determined from somite pair counts. Human fetal tissues were obtained anonymously, following spontaneous, voluntary or therapeutic pregnancy interruptions, from Magee Women Hospital, University of Pittsburgh, in compliance with Institutional Review Board protocol number 0506176. Developmental age (16 to 24 weeks of gestation) was estimated by measuring foot length. Informed consent to the use of embryonic and fetal tissues was obtained from the patients in all instances. Adult human pancreas and muscle were obtained from organ donors.

Immunohistochemistry and cytochemistry. Fresh fetal and adult tissues were gradually frozen by immersion in isopentane cooled in liquid nitrogen. Five- to 7-μm sections were cut on a cryostat (Microm), then fixed with 50% acetone (VWR International) and 50% methanol (Fischer Chemical), or for 10 min in 4% paraformaldehyde (PFA, Sigma), dried for 5 min at room temperature (RT), then washed 3 times for 5 min in phosphate-buffered saline (PBS, Gibco). Embryonic tissues were fixed for one hour at 4° C. in 4% PFA in phosphate buffer, then rinsed in PBS, impregnated in gelatin/sucrose (Sigma) and finally frozen in gelatin/sucrose in isopentane vapors, as previously described33, prior to cryosectioning. Non-specific binding sites were blocked with 5% goat serum (Gibco) in PBS for 1 hour at RT. Sections were incubated with uncoupled primary antibodies overnight at 4° C., or 2 hours at RT in the case of directly coupled antibodies. After rinsing, sections were incubated for 1 hour at RT with a biotinylated secondary antibody, then with fluorochrome-coupled streptavidin, both diluted in 5% goat serum in PBS. For intra-cellular stainings, cells were first permeabilised with PBS 0.1% Triton X-100 (Sigma).

Cultured cells were fixed inside wells with cold methanol/acetone (1:1) for 10 min, then washed 3× in PBS 0.1% Triton X-100 and incubated for 1 hour in PBS, 0.1% Triton X-100, 5% goat serum. Cultured cells were then stained as described above.

Anti-human primary antibodies used were: uncoupled CD146 (BD Pharmingen, 1:100), CD31 (DAKO, 1:100), CD34 (Serotec, 1:50), anti-NG2 (BD Pharmingen, 1:300) and anti-HNA (human nuclear antigen, Chemicon, 1:100). Coupled antibodies were: CD146-Alexa 488 (Chemicon, 1:200), anti-α-SMA-FITC (Chemicon, 1:100), CD34-FITC (DAKO, 1:50), anti-vWF-FITC (US Biological, 1:100), and biotinylated CD144 (BD, 1:100), anti-skeletal myosin heavy chain (fast) (Sigma 1:100), anti-skeletal myosin heavy chain (slow) (Sigma 1:100), anti-spectrin and anti-dystrophin (Novocastra, 1:100). Directly biotinylated Ulex europaeus lectin was also used as an endothelial cell marker (Vector, 1:200). Secondary goat anti-mouse antibodies were biotinylated (DAKO, 1:1000 and Immunotech) or coupled to Alexa 488 (Molecular Probes, 1:1000). Streptavidin-Cy3 (Sigma, 1:1000), streptavidin-Alexa 488 (Molecular Probe, 1:200), and streptavidin-peroxidase (Immunotech, 1:750) were used. To detect low amounts of surface antigens in embryonic tissues we used the TSA Plus tetramethylrhodamine amplification system (Perlin Elmer), according to manufacturer's instructions. For double-stainings, sections stained with the TSA amplification system were then incubated in 0.6% H2O2 for 10 min, washed 3 times in PBS, and exposed to an avidin-biotin blocking kit (Vector), followed by the antibody. After 3 washings, biotinylated goat anti-mouse antibody was added, followed by three washes and incubation with appropriately conjugated streptavidin. Nuclei were stained with DAPI (4′, 6-diamino-2-phenylindole dihydrochloride, Molecular Probes, 1:2000) for 5 min at RT. An isotype-matched negative control was performed with each immunostaining. Slides were mounted in glycerol-PBS (1:1, Sigma) and observed on an epifluorescence microscope (TE 2000-U, Nikon).

Flow cytometry. Pericytes present in fetal skeletal muscle, pancreas and bone marrow and in adult skeletal muscle and pancreas were analyzed and sorted by flow cytometry. Fresh pancreas or muscle tissue was cut into small pieces with a scalpel in Dulbecco's modified Eagle medium (DMEM, Gibco) containing 20% fetal bovine serum (FBS, Gibco), 1% penicillin- streptomycin (PS, Gibco) and collagenases I, II and IV (1 mg/mL, Sigma), then incubated at 37° C. for 1 hour. Final cell dissociation was achieved between ground glass slides. Cells were washed with PBS 1 mM EDTA (Gibco) and centrifuged at 1200 rpm for 5 min at 4° C. Cell pellets were resuspended in DMEM, 20% FBS, 1% PS and filtered at 100 μm (BD Falcon) in the same medium. Cells were counted following dead cell exclusion with Trypan blue (Sigma). Cells (10⁵ for analysis and around 30.106 for sorting) were incubated with one of the following directly coupled mouse anti-human antibodies: CD34-PE (DAKO, 1:100), CD45-APC-Cy7 (Santa Cruz Biotechnologies, 1:200), CD56-PE-Cy7 and CD146-FITC (Serotec, 1:100) in 1 ml DMEM, 20% FBS, 1% PS, 1mM EDTA at 4° C. for 15 min. After washing and centrifugation, cells were incubated for 30 min with 7-amino-actinomycin D (7-AAD, 1:100, BD) for dead cell exclusion, then run on a FACSAria flow cytometer (Becton-Dickinson). As negative controls, cell aliquots were incubated with isotype-matched mouse IgGs conjugated to PE (Chemicon, 1:100), APC-Cy7 (BD, 1:100), PE-Cy7 and FITC (US Biological, 1:100) in the same conditions.

RT-PCR. Total RNA was extracted from 10⁴ sorted pericytes or unfractionated cells using Trizol (Invitrogen). cDNA was synthesized with SuperScript™ II reverse transcriptase (Invitrogen), according to manufacturer's instructions. PCR was performed with Taq polymerase (Invitrogen) per manufacturer's instructions and PCR products were electrophoresed on agarose gels. The primers used for PCR are identified in the accompanying Sequence Listing. Each set of oligonucleotides was designed to spam two different exons so that genomic DNA contamination is of no concern.

Pericyte long-term culture. Pericytes sorted from skeletal fetal muscle, fetal pancreas or adult muscle were seeded at 2×10⁴ cells per cm² in EGM-2TM medium (Cambrex BioScience Inc.) and cultured at 37° C. for 2 weeks in plates coated with 0.2% gelatin (Calbiochem). Confluent cells were then detached by treatment with trypsin-EDTA (Gibco) for 15 min at 37° C., then split 1:3 in uncoated plates in DMEM high glucose (Gibco), 20% FBS, 1% PS (Gibco). After the fifth passage 1:3, cells were then passaged 1:10 in the same conditions, and culture medium was changed every 4 days.

Myogenesis in vitro. Freshly sorted or cultured pericytes (4000 and 5×10⁴ cells per cm², respectively) were cultivated in the presence of MS5 stromal cells cultured prealably in uncoated plates in a MEM, 10% FBS, 1% PS. Pericytes were co-cultured for 8-10 days in proliferation medium: 78.5% DMEM high-glucose, 10% FBS, 10% HS (horse serum, GIBCO), 5% CEE (chicken embryo extract, Accurate), 1% PS, then 5-10 days in fusion medium: 96.5% DMEM high-glucose, 1% FBS, 1% HS, 0.5% CEE, 1% PS (Gibco). Half of the medium was renewed every 4 days.

Myogenesis in vivo. Eight- to 12-week old SCID-NOD mice were used, that were anaesthetized by inhalation of isofluorane/O2. Cardiotoxin (1.5 μg/μl) was injected into the muscle one to 3 hours prior to cell transplantation. Freshly sorted or cultured pericytes suspended in 35 μl PBS were then injected into the injured muscle. Mice were sacrificed 3 weeks after transplantation and muscle was harvested for immunohistochemistry analysis.

In performance of these studies, pericytes were sorted from twenty-seven fetal (17-23 weeks of gestation) and 5 adult skeletal muscles (50-78 years) processed independently by using multi-color fluorescence-activated cell sorting (FACS). Hematopoietic cells were first gated out, as were CD56+cells in order to avoid contamination by regular myogenic progenitor cells. Pericytes were sorted on CD146 expression and lack of CD34, in order to ascertain the absence of endothelium and satellite cells18 within sorted cells. Sorted CD146+ CD34− CD45− CD56− cells, which amounted for 0.88±0.18% and 0.29±0.09% of the starting fetal and adult skeletal muscle cell populations, respectively, were indeed confirmed by RT-PCR analysis, in each cell sorting experiment, not to include hematopoietic, endothelial and regular myogenic cells. RT-PCR was also used to verify the absence of Pax7 expression by purified pericytes.

Pericytes sorted from four distinct fetal skeletal muscles were assayed for myogenic potential in culture. Sorted pericytes were cultured in the presence of MS5 mouse stromal cells, for 8-10 days in muscle proliferation medium, then for 5-10 more days in muscle fusion medium. In three out of four cases, typical myotubes containing 3-5 nuclei appeared after 8-10 days and further developed, containing up to 15 nuclei 10 days later. All myotubes developed in these conditions from sorted pericytes expressed human myosin heavy chain. To confirm and further document myogenic potential, pericytes sorted by FACS from human muscle were injected into the skeletal muscles of immunodeficient SCID-NOD mice that had been injured by intramuscular injection of cardiotoxin. Three weeks later, human spectrin immunodetection on recipient muscle sections revealed the presence of human myotubes, contrasting with sections from muscles that had only received PBS. These results confirmed that pericytes sorted stringently from human skeletal muscle are endowed with myogenic potential.

In three independent experiments, the myogenic in vivo myogenic potentials of freshly sorted pericytes (CD146+ CD45− CD34− CD144− CD56−) was quantitatively compared to that of myoblasts (CD146− CD45− CD34− CD144− CD56+) and total unseparated skeletal muscle cells. These three populations generated respectively, after injection into the cardiotoxin injured muscles of immunodeficient mice, 20.1±11.9, 13.3±5.7 and 3.0±2.5 myofibers per myofibers per 10³ cells injected.

Sorted muscle pericytes seeded in culture adhered and proliferated, and cultures could be maintained for at least 5 months. When tested at different passages, 100% of cultured pericytes still expressed CD146and α-SMA, confirming their pericyte identity. At confluence, cultured pericytes reproducibly formed-discrete spheres resembling the embryoid bodies produced in culture by embryonic stem cells. Importantly, long-term cultured muscle pericytes remained capable of differentiating into myofibers in vitro, and to robustly regenerate cardiotoxin-injured skeletal muscles in immunodeficient mice. Also, pericyte cultures containing the above-mentioned spheres have a stronger myogenic potential in vivo, quantitatively, than those in which only monolayered adherent cells are present.

### EXAMPLE 2

This example demonstrates the potential of sorted adipose-derived human pericytes to generate muscle tissue. The pericytes displayed a superior muscle regenerative ability among different groups of adipose-tissue derived stromal cells. In addition, pericytes have the ability to extensively multiply in vitro, while retaining their myogenic potency in vivo. Therefore, adipose-derived pericytes are an ideal source of autologous cells for the treatment of muscle disorders.

### Methods

Human tissues. Whole adipose tissue from abdominal subcutaneous fat, to distinguish from lipoaspirates, was obtained anonymously from patients who underwent abdominoplasty at the Department of Surgery at the University of Pittsburgh Medical Center. Donors were females from 42 to 57 years old, with a mean age of 51 years. The procedure was approved by the Institutional Review Board and the research protocol was reviewed and approved by the Animal Research and Care Committee at the Children's Hospital of Pittsburgh and University of Pittsburgh. Human fetal tissues, 20-22 weeks of gestation, were obtained anonymously, following voluntary or therapeutic pregnancy interruptions, from Magee Women Hospital, in compliance with Institutional Review Board protocol number 0506176. Informed consent to the use of fetal tissues was obtained from the patients in all instances.

Immunohistochemistry. Adipose tissues were incubated overnight in phosphate buffer, then rinsed in phosphate buffered saline (PBS), impregnated in gelatin/sucrose, and then frozen in isopentane vapors, as previously described (29), prior to cryosectioning. Cryosections of 9-μm were fixed in a 1:1 cold acetone/methanol mixture for 5 min and preincubated in 5% goat serum in PBS for 1 h at room temperature (RT). Sections were then incubated with uncoupled primary antibodies overnight at 40° C., or 2 hours at RT in the case of directly coupled antibodies. After rinsing, sections were incubated for 1 h at RT with a biotinylated secondary antibody, then with fluorochrome-coupled streptavidin. Anti-human primary antibodies used were: CD34 (Serotech, 1:50), biotinylated CD144 (Becton-Dickinson, 1:100), and CD146-Alexa 488 (Chemicon, 1:200). Secondary biotinylated goat anti-mouse (DAKO, 1:1000 and Immunotech) was followed by Cy3-conjugated streptavidin (Sigma, 1:1000).

Flow cytometry. Adult adipose tissues or fetal muscles were finely minced, then digested in Dulbecco's modified Eagle medium (DMEM, Gibco) supplemented with 3.5% bovine serum albumin (Sigma) and collagenase II (1 mg/ml, Sigma) for 75 min on a shaker at 370 C. Mature adipocytes were separated by centrifugation at 1800 rpm for 10 min and discarded. Pellets were resuspended in erythrocyte lysis buffer (155 mM NH4Cl, 10 mM KHCO3, 0.1 mM EDTA) and incubated for 10 min at RT. After washing, the cells were filtered by 70-μm cell strainer (BD Falcon). Adipose-derived cells were incubated with one of the following directly coupled mouse anti-human antibodies: CD146-FITC (Serotec, 1:100), CD34-PE (DAKO, 1:100), and CD45-APC-Cy7 (Santa Cruz Biotechnologies). As negative controls, cell aliquots were incubated with isotype-matched mouse IgGs conjugated to FITC (US Biological, 1:100), PE (Chemicon, 1:100) and APC-Cy7 (BD, 1:100) in the same conditions. Muscle-derived cells were incubated with an uncoupled anti-CD56 antibody (BD, 1:100), followed by goat anti-mouse-PE (DAKO, 1:1000). After washing and centrifugation, cells were incubated with 7-amino-actinomycin D (7-AAD, 1:100, BD) for dead cell exclusion, and then subjected to a FACSAria flow cytometer (BD).

RT-PCR. Total RNA was extracted from 2×10⁴ sorted cells using Trizol (InVitrogen). cDNA was synthesized with SuperScript™ II reverse transcriptase (InVitrogen), according to manufacturer's instructions. PCR was performed with Taq polymerase (Gibco), and PCR products were electrophoresed on agarose gels. The primers used for PCR are listed in the accompanying Sequence Listing. Each set of oligonucleotides was designed to span two different exons to exclude genomic DNA contamination.

Cell culture and myogenic differentiation in vitro. Sorted adipose-derived cells were seeded at an initial density of 2×10⁴ cells per cm² in DMEM containing 20% fetal bovine serum (FBS) and 1% penicillin-streptomycin (all GIBCO-brand reagents from Invitrogen, Carlsbad, Calif.). Sorted muscle-derived cells were seeded at an initial density of 2×10⁴ cells per cm2 in proliferation medium (DMEM 10% FBS, 10% horse serum, 1% penicillin/streptomycin, 1% chick embryo extract; GIBCO-BRL). At 70% confluence, cells were detached with trypsinIEDTA, replated after washing at densities between 2.0 to 3.0×10³ cells per cm², and further cultured for up to 5 months for fat-derived cells. Muscle-derived cells were differentiated into myotubes by switching to a fusion medium (DMEM 2% FBS, 1% penicillin/streptomycin).

Immunostaining of long-term cultured pericytes. Cultured cells were fixed, with cold methanol/acetone for 10 min, washed in PBS, and incubated for 1 hour in PBS with 5% goat serum. Cells were then stained with the following antibodies: uncoupled CD146 (BD Pharmingen, 1:100), uncoupled NG2 (BD Pharmingen, 1:300) and αSMA-FITC (Chemicon, 1:100). Uncoupled antibodies were revealed with biotinylated goat anti-mouse antibody (DAKO, 1:1000), followed by streptavidin Cy3 (Sigma, 1:1000).

Myofiber regeneration in vivo. A total of 1×10⁴ sorted and unfractionated stromal cells from adipose tissues of 5 patients, sorted CD56+ cells from muscles of 2 fetuses, or 2 independent samples of adipose-derived pericytes which had been cultured for a period of 16 weeks, were injected into the gastrocnemius muscle of female NOD-SCID mice (6 to 8 weeks old) which had been injured by intramuscular injection of 15 μl of 50 μM cardiotoxin (Sigma) 2 hours earlier. Eighteen to twenty days after transplantation, the gastrocnemius muscles were harvested, flash frozen in liquid nitrogen-cooled 2-methylbutane, and serially sectioned (9 μm). Spectrin staining was performed on goat serum-blocked sections using a human-specific anti-β-spectrin mouse antibody (1:100; Novocastra) to detect human cell derived myofibers. Sections were then washed in PBS and incubated with a biotinylated anti-mouse IgG antibody, followed by washing and incubation with Cy3-streptavidin (Sigma).

Population doubling time in culture. Sorted cells cultured in DMEM+20% FBS for 10 weeks were seeded into 6-well plates at a density of 2.0×103 cells/cm2. Cells were grown for 120 hours, and the population doubling time was calculated using the formula: time/no. of doublings, where time=120, and no. of doublings=log2 (N final/N initial).

Preplating of adipose stromal cells. The total, freshly isolated stroma vascular fraction was plated in DMEM+20% FBS. After one and a half hours, cells which did not adhere to the flask were collected and named NC. Cells attached to the flask at this time were also collected and named AC. The NC and AC fractions were labeled separately for FACS analysis as described above, and the number of pericytes per 1×10⁴ cells in each group was determined.

Statistical analysis. Data are expressed as the mean±SEM. An unpaired Student's t-test was used for statistical analysis, with a p value of <0.05 considered to be statistically significant.

### Results

Immunohistochemical detection of vascular cells in human adipose tissue. Adipose tissue sections were labeled with an antibody to CD34, an antigen expressed by endothelial cells but not pericytes, and CD146, also known as S-endo1, another antigen expressed by endothelial cells but also found on the surface of pericytes. Ubiquitous distribution of endothelial cells, as marked by CD34 expression, was observed within the adipose tissue. Double-staining of adipose tissue capillaries with antibodies to CD34 and CD146 shows CD34-negative CD146-positive pericytes in a typical perivascular location, closely adherent to endothelial cells within capillaries. The expression of CD146 by pericytes was also observed in arterioles and venules, rarely seen in adipose tissue sections, with pericytes assuming an abluminal location within the blood vessel wall.

Flow cytometry sorting of human adipose-derived vascular cells. Following enzymatic dissociation of whole adipose tissue, the stromal vascular cells (SVC) were separated from adipocytes by centrifugation. On average, 3.5×10⁵ SVC were obtained per gram of adipose tissue, while the maximum cell yield was 1×10⁶ SVC. In order to separate pericytes from endothelial cells by FACS sorting, freshly isolated stromal cells were labeled with antibodies directed against CD34, CD45 and CD146. Hematopoietic lineage cells, which express CD45 and accounted for 5.59±1.48% of the starting cell population, were first excluded, and the remaining cells were further analyzed for CD34 and CD146 expression. From this analysis, four distinct adipose-tissue derived, non-hematopoietic cell populations were delineated and sorted:

The first population consists of CD34+CD146− CD45− cells, suggesting that it is composed of vascular endothelial cells or endothelial progenitor cells, herein named EC. The second population consists of CD34+CD 146+ CD45− endothelial cells, herein named S-endo1+ endothelial cells (S-EC). Two subsets of endothelial cells were thus observed based on S-endo1 expression. EC and S-EC accounted for the majority of cells within the stromal cell fraction and comprised 67.5±2.42% and 10.1±1.17% of this fraction, respectively. The third population consists of CD34− CD146+ CD45− cells, suggesting they correspond to pericytes (PC). Pericytes accounted for 14.6±1.02% of the stromal cells. An approximate ratio of pericytes to endothelial cells of 1:6 is thus observed in the adipose tissue. The last cell population consisted of CD34− CD146− CD45− cells, which may correspond to non-vascular cells (NVC) and amounted to 7.8±2.29% of the total stromal cell fraction.

To test the efficiency of our sorts, RT-PCR analyses of the three sorted vascular cell populations was performed. The results demonstrated a good separation of EC, S-EC and PC based on differential expression of CD34 and CD146. Notably, sorted pericytes were free of contaminating CD34+ endothelial cells. To confirm the identity of the sorted cells, the expression of desmin and von Willebrand factor (vWF) (which are established pericyte and endothelial cell markers, respectively) was assessed. The sorted pericytes were found to express desmin, while the two endothelial cell populations expressed vWF. Pure populations of endothelial cells not contaminated by pericytes also were obtained, as both EC and S-EC did not express desmin. Likewise, PC did not express vWF, confirming that there was no contamination of sorted pericytes by endothelial cells. RT-PCR analysis also showed that the two sorted endothelial cell populations, EC and S-EC, differ from each other with regard to NG2 expression, which is generally considered a pericyte marker but has also been found expressed by proliferating endothelial cells involved in angiogenesis.

Pericytes are superior to other stromal cells in regenerating muscle fibers in vivo. Freshly sorted cells were transplanted into the injured gastrocnemius muscles of NOD-SCID mice. Three weeks after implanting the cells, muscle regeneration by immunodetection of human spectrin on frozen sections of treated muscles was analyzed. Human spectrin-expressing muscle fibers were observed in each group. However, the regeneration index, a measure of the number of myofibers formed by injected cells, was significantly higher in the pericyte group than that in all other cell groups. Limited muscle regeneration was observed in the EC, S-EC, and NVC groups, and differences in regeneration indexes were not significant between these groups. To test whether transplantation of a mixed population of cells might offer an advantage in muscle regeneration over that of a homogeneous, pure cell population, we transplanted into NOD-SCID mice the same number of freshly isolated unsorted SV cells. The regenerative index of unsorted SV cells was merely comparable to that of the sorted EC, S-EC and NVC groups.

The identification of a highly myogenic cell population within adipose tissue may raise the concern of contamination with muscle tissue and the resident satellite cells contained therein. Therefore, the expression of the Pax7 transcription factor, a key marker of muscle satellite cells which are the professional myogenic progenitors, was tested in sorted hWAT pericytes. The results showed that the pericyte group sorted from adipose tissue did not contain cells expressing Pax7. Total unfractionated cells from human muscle were used as a positive control.

The pericytes with muscle satellite cells also were compared in terms of myogenic potential. The CD56+ compartment, corresponding to satellite cells and amounting to approximately 42% of the total population, was sorted from fetal skeletal muscles. Freshly sorted satellite cells spontaneously formed muscle fibers in a robust manner when placed in culture. The cells were then injected into NOD-SCID mouse skeletal muscles under the same experimental conditions used for the WAT cells. Analysis of the engrafted muscles showed the regeneration index of human satellite cells to be in the same range but not higher than that of WAT-derived pericytes.

The muscle regenerative capacity of human adipose-derived pericytes is unaffected by long-term culture. When seeded in culture, it was consistently observed that sorted pericytes took at least 5-7 days to adhere. In comparison, sorted EC rapidly attached to the flask within a few hours. Primary cultures of sorted pericytes revealed cells with a distinct morphology that is typical of pericytes, i.e. broad and flat, with irregular or ruffled edges. In contrast, the other 3 populations exhibited smooth borders. Primary cultures of EC showed fusiform cells while S-EC exhibited a round morphology. NVC appeared long and narrow. Pericyte cultures could be maintained for at least 5 months by repeated collection at 70% confluence and replating at densities between 2.0 to 3.0×10³ cells/cm². In culture, it was further observed that pericytes did not reach confluence and had a significantly longer doubling time compared to EC and NVC. Approximately 90% of long-term cultured pericytes still expressed CD146, NG2 and αSMA, confirming their pericyte identity. RT-PCR analysis of cultured pericytes further confirmed expression of CD146 and NG2. To test their myogenic potential in vitro, cultured pericytes were re-cultured for 14 days in a low-serum medium conditioned with human myofibers. Under these conditions, pericytes occasionally formed multinucleated myotubes, but at a frequency lower than that characterizing human satellite cells cultured in low-serum medium. However, long-term cultured pericytes remained capable of generating human muscle fibers in NOD-SCID mice and presented a regenerative index which was statistically in the same range as that of freshly isolated pericytes.

Selection of pericytes by preplating of adipose-derived stromal cells. A single preplating of adipose stromal cells was performed. The cell composition of adherent (AC) and non-adherent fractions (NC) was compared. FACS analysis of equal number of cells in the NC and AC fractions showed a visible difference in the number of pericytes between the two fractions, and quantification revealed the number of pericytes within the NC fraction to be four-fold higher compared to the AC fraction. Further, when each cell population was analyzed individually for adherence properties, the majority of endothelial cells, close to 70%, were fast-adhering cells. In contrast, only about 20% of pericytes and s-endo1+ endothelial cells were fast-adhering. Lastly, the non-vascular fraction consisted of approximately equal numbers of slow and fast-adhering cells. These results suggest that by means of preplating, the pericyte population can be significantly enriched in comparison with unsorted adipose stromal cells.

### EXAMPLE 3

This example demonstrates vascular pericytes serve as multipotent stem cells in human organs.

As noted in Examples 1 and 2, sorted pericytes (CD146+, NG2+, CD34−, CD31−, CD45− and CD56−) represent a population of cells with significant myogenic potential. However, this same population of pericytes produced chondrocytes, adipocytes (PPARγ+, leptin+) and neuron-like cells (NF+, TOH+, CNPase+) when cultured in the appropriate differentiation conditions, and human hematopoietic cells (CD45+, HLAC11+) when co-cultured in the presence of MS5 stromal cells or injected into irradiated SCID-NOD mice. Accordingly, pericytes prospectively sorted to homogeneity from multiple human tissues are endowed with broad developmental potential.

### EXAMPLE 4

This example demonstrates the ability of human pericytes to recover cardiac function and increase survival via tissue engineering in vivo.

Pericytes are isolated from human skeletal muscle, bone marrow or adipose tissue as CD146+ CD34− CD45− CD56− cells. Sorted cells are re-analyzed and RT-PCR is performed to verify the absence of contaminant cells. Purified pericytes are first cultured for 5 days in EGM-2 endothelial cell medium, then switched to MEM 20% FCS.

For cell transplantation and engraftment analysis, myocardial infarction is induced in anesthetized nude rats via ligation of the left anterior descending coronary artery. Human pericytes in PBS are immediately injected into the contracting wall bordering the infarct and into its center. One, 2, 6, and 12 weeks later one population of rats is sacrificed and hearts are harvested, frozen and serially cryosectioned. FISH (fluorescent in situ hybridization) of a human probe is used to track the human cells implanted in the myocardium. The same sections are used for detection of human spectrin or lamin. The sections are counterstained with DAPI to reveal all nuclei. The number of human spectrin+myofibers and the number of donor-derived nuclei are determined at different time points. Digitized images are evaluated to determine more effectively the area of engraftment within each injected heart. Anti-cardiac troponin I, anti-atrial natriuretic peptide (ANP), anti-Nkx2.5, anti-α-myosin heavy chain (α-MHC), anti-GATA-4, anti-connexin43 are used to investigate the acquisition of a myocardiac phenotype by the injected cells. Capillary density in the heart cryosections is monitored after anti-vWF, anti-CD144, anti-CD34 and anti-CD31 immunostaining, as is the expression of VEGF and VEGF receptor (KDR).

For echocardiographic assessment, 4 and 8 weeks post implantation, another population of rats is anesthetized with isoflurane and standard transthoracic echocardiography is performed. Heart rate, left ventricular (LV) dimensions and wall motion including fractional area change (FAC) and shortening fraction (SF) are measured. LV inflow velocities and time-intervals, main pulmonary artery and aortic blood flow velocity are measured to calculate the cardiac output. Arterial blood pressure is monitored during the echocardiography. Left ventricular pressure is measured on animals anesthetized with isoflurane, using a 1.7Fr pressure manometer.

Passive LV inflation tests are performed in another population of rats at 8 weeks after cell implantation. The heart is exposed and arrested by apical injection of a hypothermic and hyperkalemic solution. The heart is excised, rinsed, and coronary arteries are perfused, then occluded at the proximal site. For LV surface strain measurement, graphite particle markers are placed on the infarcted LV epicardial surface to measure surface strain at a given intra-LV pressure. Pressure is applied to the LV via a volume-infusion pump with a lured cannula and a micromanometer-tipped catheter. LV cavity pressure and LV surface markers are tracked continuously with a bronchoscope and a CCD camera to obtain pressure-regional LV surface strain relations (regional LV compliance) using a custom-made LabVIEW system.

To assess cell survival and proliferation, in another population of rats, after human cell implantation in the infarct, BrdU is injected intraperitoneally. Rats are sacrificed 24 hours later and double-labeled cells (BrdU/human lamins A/C or BrdU/human probe) within the injected hearts are tracked with an antibody against BrdU. Apoptotic cells are detected by the TUNEL reaction on heart sections and colocalized with cells expressing human lamins A/C. The fusion of donor cells with one another or with host cardiomyocytes is also assessed using standard immunohistochemistry techniques.

The results of these studies can demonstrate that the pericytes proliferate, survive, and acquire a cardiac cell phenotype (through differentiation or fusion) and also exhibit the ability to differentiate into blood vessels, secrete vascular endothelial growth factor (VEGF), and stimulate angiogenesis. Furthermore, the injection of human pericytes can achieve a function functional recovery after myocardial infarction in the treated rats.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A mammalian pericyte having a marker pattern comprising CD146+, CD34−, and CD45−, wherein said pericyte is substantially isolated from cells that are CD146− or CD31+ or CD34+ or CD45+ or CD56+ or NG2− or CD133− or a combination of one or more of such markers.

2. The pericyte of claim 1, which is CD31−.

3. The pericyte of claim 1, which is CD56−.

4. The pericyte of claim 1, which is NG2+.

5. The pericyte of claim 1, which is CD133+.

6. The pericyte of any of claims 1-5, which has one or more developmental phenotypes selected from the group of developmental phenotypes consisting of adipogenic, chondrogenic, myogenic, myocardiogenic, neurogenic, odontogenic, osteogenic, and vascular.

7. The pericyte of claim 6, which is human.

8. A substantially homogenous population of pericytes according to claim 6.

9. A population according to claim 8, which retains the developmental phenotype after culture in vitro for at least about five months.

10. A method for isolating a human pericyte, the method comprising obtaining tissue from a human donor, dissociating cells within said tissue, assaying for a pericyte which is CD146+, CD34−, and CD45−, separating said pericyte from other cells which are CD146− or CD31+ or CD34+ or CD45+ or CD56+ or NG2− or CD133− or a combination of one or more of such markers, and culturing said pericyte.

11. The method of claim 10, wherein said pericyte is CD31−.

12. The method of claim 10, wherein said pericyte is CD56−.

13. The method of claim 10, wherein said pericyte is NG2+.

14. The method of claim 10, wherein said pericyte is CD133+.

15. The method of any of claims 10-14, wherein said assaying and separation are accomplished by flow cytometry.

16. The method of claim 15, wherein said flow cytometry is fluorescence activated cell sorting (FACS).

17. A method for engineering tissue in vivo comprising obtaining tissue from a human donor source, dissociating cells within said tissue, assaying for a pericyte which is CD146+, CD34−, and CD45−, separating said pericyte from other cells which are CD146− or CD31+ or CD34+ or CD45+ or CD56+ or NG2− or CD133− or a combination of one or more of such markers, and introducing said pericyte into a recipient subject at a location for the pericyte to generate or repair tissue within the recipient subject.

18. The method of claim 17, wherein said pericyte is CD31−.

19. The method of claim 17, wherein said pericyte is CD56−.

20. The method of claim 17, wherein said pericyte is NG2+.

21. The method of claim 17, wherein said pericyte is CD133+.

22. The method of claim 17, wherein the recipient is the same as the donor source.

23. The method of claim 17, wherein the tissue is selected from the group of tissues consisting of fat, muscle, cartilage, bone, and vasculature.

24. The method of claim 17, wherein the tissue is myocardium.

25. The method of claim 17, wherein the recipient is human.

26. The method of claim 17, wherein the donor source is an embryo or placental.

27. A method for engineering tissue in vivo comprising introducing the population of claim 7 into a recipient subject at a location for the pericyte to generate or repair tissue within the recipient subject.

28. The method of claim 27, wherein the tissue is selected from the group of tissues consisting of fat, muscle, cartilage, bone, and vasculature.

29. The method of claim 27, wherein the tissue is myocardium.

30. The method of claim 27, wherein the recipient is human.


SEQUENCE LISTING (32 sequences)

SEQ ID NO: 1 - DNA, 22 bp, Artificial
  Sense primer for CD45 used in Example 1
catgtactgctcctgataagac

SEQ ID NO: 2 - DNA, 21 bp, Artificial
  Antisense primer for CD45 used in Example 1
gcctacacttgacatgcatac

SEQ ID NO: 3 - DNA, 21 bp, Artificial
  Sense primer for a-SMA used in Example 1
ttccttcgttactactgctga

SEQ ID NO: 4 - DNA, 21 bp, Artificial
  Antisense primer for a-SMA used in Example 1
cgatccagacagagtatttgc

SEQ ID NO: 5 - DNA, 21 bp, Artificial
  Sense primer for CD146 used in Example 1
aaggcaacctcagccatgtcg

SEQ ID NO: 6 - DNA, 21 bp, Artificial
  Antisense primer for CD146 used in Example 1
ctcgactccacagtctgggac

SEQ ID NO: 7 - DNA, 22 bp, Artificial
  Sense primer for vWF used in Example 1
gcaccattcagctaagaggagg

SEQ ID NO: 8 - DNA, 22 bp, Artificial
  Antisense primer for vWF used in Example 1
gcttcccaccttgacatactgc

SEQ ID NO: 9 - DNA, 20 bp, Artificial
  Sense primer for CD144 used in Example 1
gatgcagaggctcatgatgc

SEQ ID NO: 10 - DNA, 20 bp, Artificial
  Antisense primer for CD144 used in Example 1
gatgctgtacttggtcatcc

SEQ ID NO: 11 - DNA, 22 bp, Artificial
  Sense primer for CD34 used in Example 1
catcactggctatttcctgatg

SEQ ID NO: 12 - DNA, 21 bp, Artificial
  Antisense primer for CD34 used in Example 1
agccgaatgtgtaaaggacag

SEQ ID NO: 13 - DNA, 18 bp, Artificial
  Sense primer for beta-Actin used in Example 1
cctcgcctttgccgatcc

SEQ ID NO: 14 - DNA, 20 bp, Artificial
  Antisense primer for beta-Actin used in Example 1
ggaatccttctgacccatgc

SEQ ID NO: 15 - DNA, 21 bp, Artificial
  Sense primer for CD56 used in Example 1
gtatttgcctatcccagtgcc

SEQ ID NO: 16 - DNA, 22 bp, Artificial
  Antisense primer for CD56 used in Example 1
catacttcttcacccactgctc

SEQ ID NO: 17 - DNA, 21 bp, Artificial
  Sense primer for NG2 used in Example 1
gtctacgctgggaatattctg

SEQ ID NO: 18 - DNA, 20 bp, Artificial
  Antisense primer for NG2 used in Example 1
ctggcccacgaaagtggaag

SEQ ID NO: 19 - DNA, 22 bp, Artificial
  Sense primer for CD34 used in Example 2
catcactggctatttcctgatg

SEQ ID NO: 20 - DNA, 21 bp, Artificial
  Antisense primer for CD34 used in Example 2
agccgaatgtgtaaaggacag

SEQ ID NO: 21 - DNA, 21 bp, Artificial
  Sense primer for CD146 used in Example 2
aaggcaacctcagccatgtcg

SEQ ID NO: 22 - DNA, 21 bp, Artificial
  Antisense primer for CD146 used in Example 2
ctcgactccacagtctgggac

SEQ ID NO: 23 - DNA, 21 bp, Artificial
  Sense primer for NG2 used in Example 2
gtctacgctgggaatattctg

SEQ ID NO: 24 - DNA, 20 bp, Artificial
  Antisense primer for NG2 used in Example 2
ctggcccacgaaagtggaag

SEQ ID NO: 25 - DNA, 22 bp, Artificial
  Sense primer for vWF used in Example 2
gcaccattcagctaagaggagg

SEQ ID NO: 26 - DNA, 22 bp, Artificial
  Antisense primer for vWF used in Example 2
gcttcccaccttgacatactgc

SEQ ID NO: 27 - DNA, 20 bp, Artificial
  Sense primer for Desmin used in Example 2
gaagtgaaccggctcaaggg

SEQ ID NO: 28 - DNA, 20 bp, Artificial
  Antisense primer for Desmin used in Example 2
cgagctagatgagctgcatc

SEQ ID NO: 29 - DNA, 20 bp, Artificial
  Sense primer for PAX7 used in Example 2
accaggagaccgggtccatc

SEQ ID NO: 30 - DNA, 19 bp, Artificial
  Antisense primer for PAX7 used in Example 2
cccgaacttgattctgagc

SEQ ID NO: 31 - DNA, 18 bp, Artificial
  Sense primer for beta-Actin used in Example 2
cctcgcctttgccgatcc

SEQ ID NO: 32 - DNA, 20 bp, Artificial
  Antisense primer for beta-Actin used in Example 2
ggaatccttctgacccatgc